(19) United States
(12) Patent Application Publication
(10) Pub. No.: US 2004/0092801 A1
(43) Pub. Date: May 13, 2004

(54) System for, and method of, acquiring physiological signals of a patient

(76) Inventor: Budimir Drakulic, Los Angeles, CA (US)

Correspondence Address: ELLSWORTH R. ROSTON, ESQ., FULWIDER PATTON LEE & UTECHT, LLP, HOWAR HUGHES CENTER, TENTH FLOOR, 6060 CENTER DRIVE, LOS ANGELES, CA, 90018, US

(21) Appl. No.: 10/293,105

(22) Filed: Nov. 13, 2002

(51) Int. Cl. A61B 5/00

(52) U.S. Cl. 600/300

(57) ABSTRACT

A host directs a microprocessor to command each of a plurality of amplifiers to process signals relative to individual ones of a plurality of physiological signals in a patient. The signals are provided to the amplifiers by terminals which are connected to different parts of the patient's body. The terminals provide signals to the amplifiers simultaneously but the microcompressor processes the signals sequentially. The microprocessor tests and calibrates the amplifiers before processing the signals from the terminals. The amplifiers have substantially the same construction regardless of where the associated terminals are disposed on the patient's body. The amplifiers may be provided with characteristics to eliminate noise and to provide output signals in a limited frequency range in which the relative phases of the signals in the limited frequency range are preserved.

This invention relate to systems for, and methods, of measuring individual physiological signals of a patient. The invention particularly relates to a system for, and a method of, measuring such physiological signals on a more precise and automated basis than in the prior art.

### BACKGROUND OF THE INVENTION

Systems are known in the prior art for acquiring physiological signals of a patient. In such systems, a transducer is attached to different external positions on the patient dependent upon the physiological signals to be acquired. For example, terminals may be attached to particular external positions on a patient's head and body to determine whether the patient has a sleep apnea and, if so, what is causing the sleep apnea. As another example, terminals are attached to particular external positions around a patient's torso to determine whether the patient is having, or has had, a heart attack.

The signals from the terminals generally are characterized by an amplitude and frequency band dependent upon the transducer type, transducer position, patient's health status and measurements that are being made. The frequency of the signals from the terminals generally are characterized to have a frequency bandwidth in a range from DC to less than approximately one hundred hertz (100 Hz) and an amplitude in a range from a few microvolts to several millivolts. For example, the signal may have a frequency in the range of approximately 50 hertz when a patient's eye movements are measured to determine sleep apnea and the signals from the terminals may have a frequency in the range to approximately 1 hertz when the galvanic skin response is measured.

Different systems are now in use for measuring the characteristics of signals from terminals disposed at strategic external positions on a patient. For example, one system provides for sleep recordings and other systems provide 12-lead electrocardiogram measurements. However, there is no single system operating to provide different types of measurements such as sleep apnea and 12-lead electrocardiograms in one setting. This prevents comparisons and correlations between the signals produced at different terminals from being accurate.

Furthermore, the systems now in use do not respond automatically to instructions from a host for sequentially setting up, calibrating and testing the response of amplifiers in the different channels in the system to the signals from the different ones of the terminals. This is particularly true when changes have had to be made in the initial operating characteristics of the different amplifiers because the initial operating characteristics for the amplifiers do not provide an optimal output. Another disadvantage has been that, although amplifiers are provided, each to respond to the signals from an individual one of the terminals, each amplifier has had a different construction and characteristics from the other amplifiers because of the individual characteristics of the signals introduced to the amplifier.

### BRIEF DESCRIPTION OF A PREFERRED EMBODIMENT OF THE INVENTION

A host directs a microprocessor to command each of a plurality of amplifiers to process signals relative to individual ones of a plurality of physiological signals in a patient. The signals are provided to the amplifiers by terminals which are connected to different parts of the patient's body. The terminals provide signals to the amplifiers simultaneously but the microcompressor processes the signals sequentially. The microprocessor tests and calibrates the amplifiers before processing the signals from the terminals. The amplifiers have substantially the same construction regardless of where the associated terminals are disposed on the patient's body. The amplifiers may be provided with characteristics to eliminate noise and to provide output signals in a limited frequency range in which the relative phases of the signals in the limited frequency range are preserved.

### BRIEF DESCRIPTION OF THE DRAWINGS

In the drawings:

FIG. 1 is a schematic perspective view of a patient with terminals applied to the patient to make tests such as electrocardiography and electroencephalography tests on the patient;

FIG. 2 shows an electrical system, primarily in block form, of programmable solid state recorder (PSSR) including a plurality of amplifiers for providing different physiological signal measurements, such as electrocardiography and electroencephalography measurements, on the patient on a closed loop basis where the closed loop provides for corrections to obtain optimal measurements on the patient;

FIG. 3 is a flow chart showing the successive steps provided by the system shown in FIG. 2 to obtain the optimal measurements;

FIG. 4 is an electrical circuit diagram, primarily in block form, showing on a schematic basis the construction and operation of one of the amplifiers shown in FIG. 2;

FIG. 5 is a circuit diagram, primarily in block form, showing the interrelationship between a pair of recorders, each indicating the output from an individual one of the PSSR shown in FIG. 2, and a central archive for storing the data and further indicating the coupling of the recorders and the central archive through a high speed digital subscriber line (DSL);

FIG. 6 is a circuit diagram, primarily in block form, showing the intercoupling of recorders and the central archive through a high speed wide area network (WAN) on a wireless area network basis;

FIG. 7 is a circuit diagram, primarily in block form, showing the intercoupling of one of the recorders and the central archive through a DSL and showing the intercoupling of other recorders and the central archive through a high speed wide area network on a wireless basis; FIGS. 8-1 and 8-2 provide a detailed circuit diagram setting forth the construction in detail of one of the amplifiers shown in block form in FIG. 4;

FIG. 9A and 9B constitute a chart showing different types of physiological signals capable of being measured on the patient and the individual characteristics distinguishing these different physiological signals;

FIG. 10 is a simplified diagram of one of the circuits included in the amplifier of FIG. 8 and shows one of the impedances whose value is changed in accordance with differences in one of the characteristics desired for the output from the circuit;

FIG. 11 is a chart showing how the output of the circuit shown in FIG. 10 varies in accordance with changes in the value of the impedance in FIG. 10;

FIG. 12 is a diagram of another one of the circuits included in the amplifier of FIG. 8 and show another one of the impedances whose value is changed in accordance with differences in another one of the characteristics desired for the output from the circuit; and

FIG. 13 is a chart showing how the output of the circuit shown in FIG. 12 varies in accordance with the changes in the value of the impedance in FIG. 12; and

FIG. 14 is a detailed circuit diagram, similar to that shown in FIG. 8, of one of the amplifiers and includes a multiplexer for selecting the amplifier from among the other amplifiers in the system.

### DETAILED DESCRIPTION OF A PREFERRED EMBODIMENT OF THE INVENTION

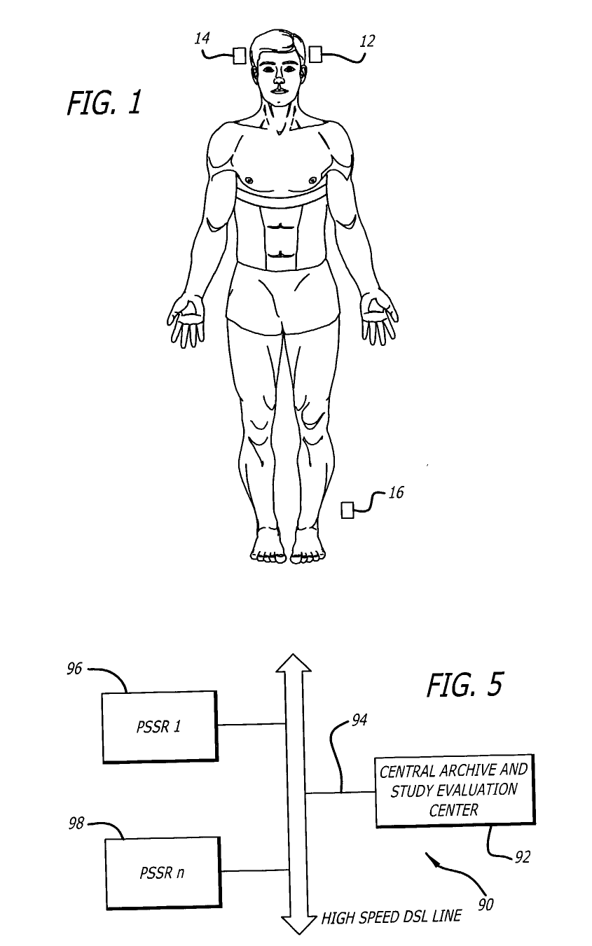

FIG. 1 is a schematic diagram of a system, generally indicated at 10, for producing signals at strategic external positions on a patient's body. For example, the system 10 may include terminals or electrodes 12 and 14 which may be applied to strategic positions on a patient's head to determine signals produced by the patient's brain at these strategic positions while the patient is asleep. These signals, with other terminals providing sleep studies, may be analyzed to determine if the patient has sleep apnea and, if so, to determine what causes the patient's sleep apnea. It will be appreciated that the terminals or electrodes 12 and 14 are illustrative only. For example, a terminal 16 may be applied to one of the patient's legs to help determine another signal in evaluating patient's sleep apnea.

Terminals, electrodes or transducers may also be applied to the patient's body at other strategic positions on the patient's body. The terminals, electrodes and transducers may provide signals indicative of other physiological signals from the patient's body. For example, terminals, electrodes or transducers may be applied to (1) the patient's scalp to determine or measure the patient's electroencephalography (EEG), (2) above the patient's eye to determine the patient's electrooculography (EOG) and to the patient's face or legs to determine the patient's electromyography (EMG).

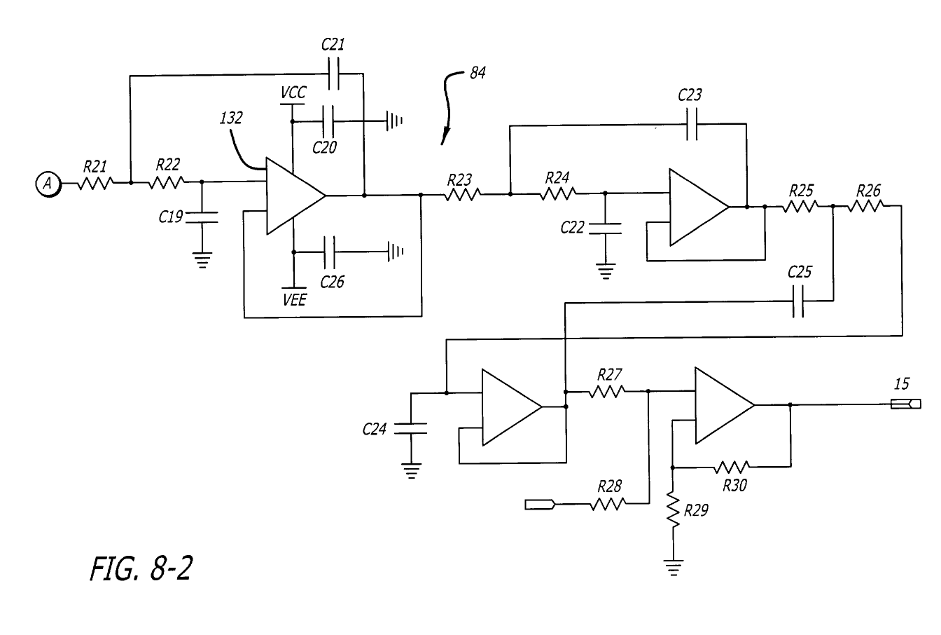

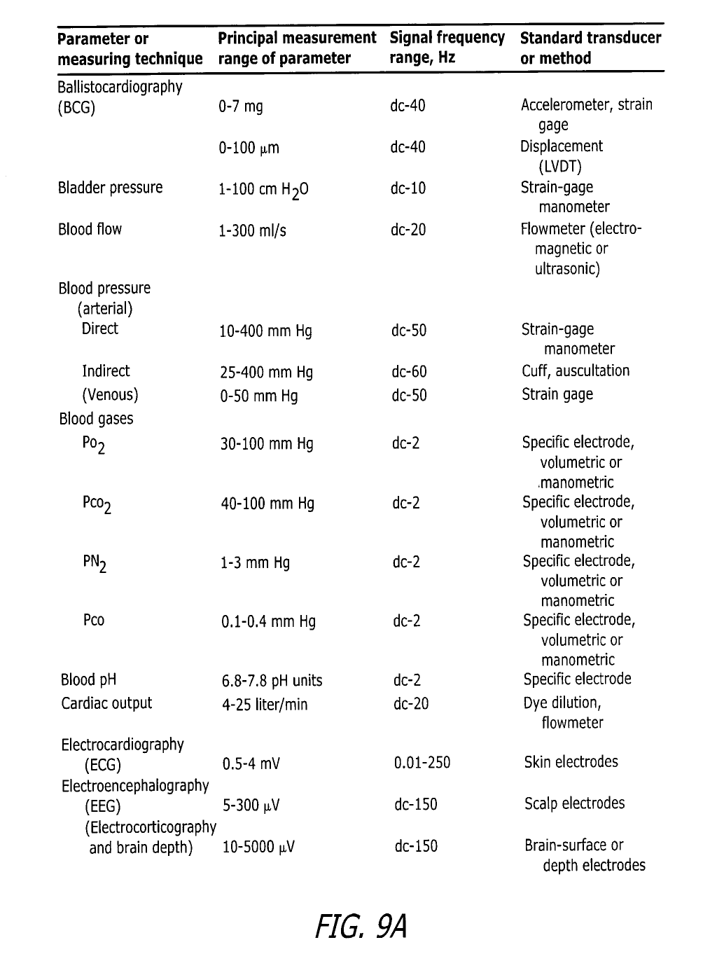

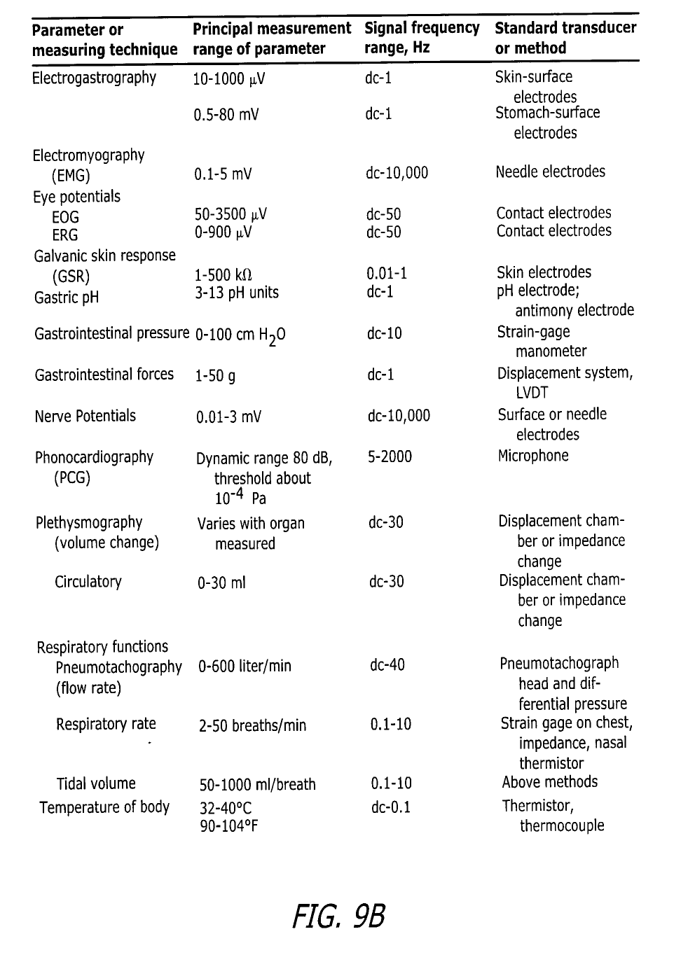

The signals produced at the different terminals such as the terminals 12, 14 and 16 have different characteristics dependent upon where the terminals are located on the patient's body. One of the different characteristics may be the range of frequencies of the signals produced at the different terminals. This range of frequencies may be specified in the third (3^{rd}) column in the chart shown in FIGS. 9A and 9B for the physiological signals specified in the first column in FIGS. 9A and 9B. For example, the range of frequencies may be from direct current (DC) to 50 Hertz for blood pressure, may be from DC to 20 hertz for cardiac output and may be from DC to 250 hertz for electrocardiography. The chart shown in FIGS. 9A and 9B is obtained from a book entitled Medical Instrumentation, edited by Webster and has been revised in that book from Medical Engineering and has been edited by C. D. Rayard and copyrighted in 1974 by Year Book Medical Publishers, Inc. of Chicago, Ill.

The type of output, and the amplitude range of each type of output, for each parameter is specified in the second column of FIGS. 9A and 9B. For example, the measurement of electrocardiography may be from 0.5 millivolts to 4 millivolts and the measurements of electroencephalography may be from 5 microvolts to 300 microvolts. The fourth column in FIGS. 9A and 9B specifies the standard transducer or method used to measure each physical parameter on or in the patient's body. As will be appreciated, a considerable number of the different physiological signals are measured by applying terminals or electrodes externally to the patient's body. However, other physiological signals involve transducers other than terminals or electrodes. Because of this, when the term “terminal” is used in the specifications and the claims, it is intended to cover all of the different transducers, electrodes and methods specified in the fourth column of FIGS. 9A and 9B.

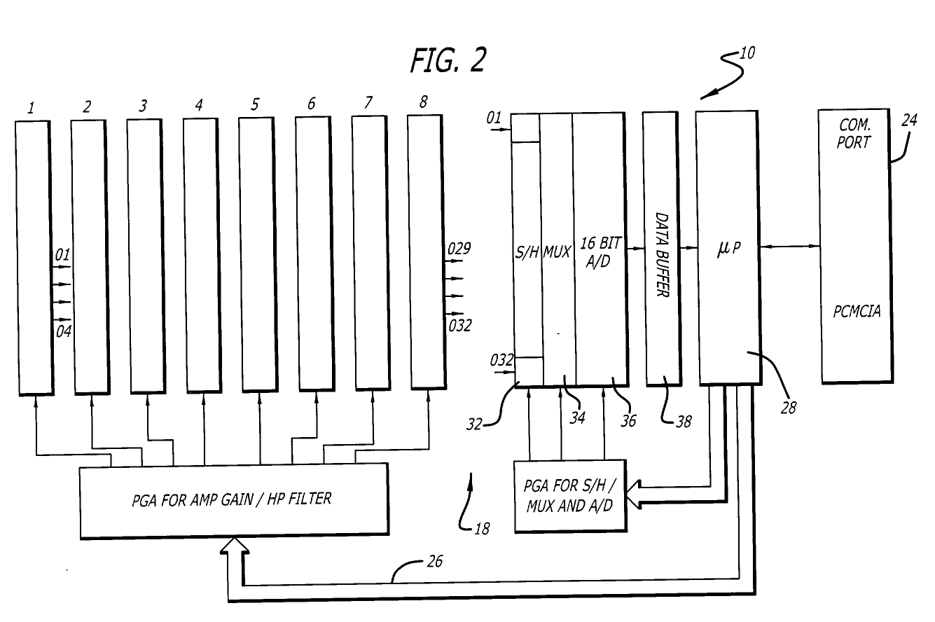

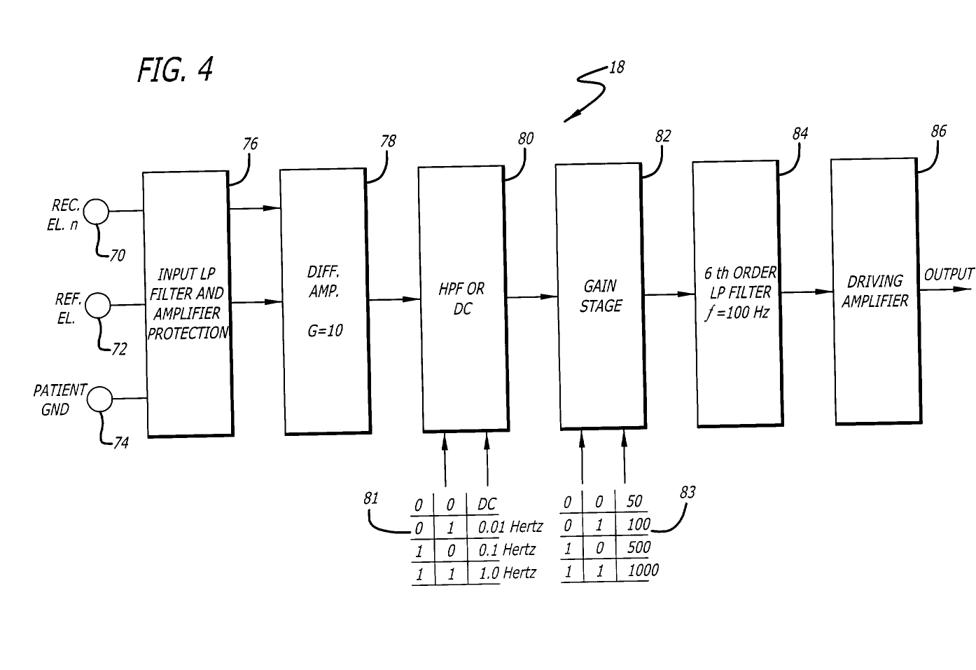

FIG. 2 is a schematic diagram, primarily in block form, of the system 10 in which eight (8) blocks 18 are provided and in which four (4) amplifiers, each generally indicated at 18 in FIG. 4, are provided in each block as illustrated by outputs 0-4 from the first block and outputs 29-32 from the eighth (8^{th}) block. The thirty-two (32) amplifiers are disposed in thirty-two (32) channels and are identical. However, a first impedance is provided in one stage in each amplifier, with four (4) alternative values to provide an adjustment in the minimum frequency of the signals from that stage. A second impedance is provided in a second stage of the amplifier with four (4) alternative values to provide an adjustment in the gain in that stage so as to maintain the gain of the stage between particular maximum and minimum limits.

In spite of the fact that each of the amplifiers has the same construction except for the four (4) alternative values in each of the first and second impedances in each of the amplifiers, each amplifier is able to provide an operative and reliable output regardless of the physiological signal which the amplifier is instructed by a host to measure. The provision of a single amplifier construction regardless of the individual one of the physiological signals being measured by the amplifier offers certain advantages. One advantage is that the standardization of the amplifiers simplifies the construction of the amplifiers and simplification is generally an advantage. Another advantage is that a user can select any amplifier to determine any physical parameter without any concern that he will obtain improper measurements if he or she selects the wrong amplifier to measure an individual one of the physiological signals. The differences between the measurements of individual ones of the physiological signals are resolved by selecting the individual ones of the four values of the first and second adjustable impedances dependent upon the physiological signals to be measured by the amplifiers.

In the system 10 shown in FIG. 2, a host 24 is provided for instructing the microprocessor in each PSSR to adjust the amplifier through a bus 26 in how to operate in measuring individual ones of the physiological signals of the patient. These instructions are introduced to a microprocessor 28 which then instructs each amplifier how the amplifier is to operate to measure an individual one of the physiological signals of the patient. For example, these parameters may relate to sleep apnea or to electrocardiography or to electroencephalography. These instructions may involve the value of the first impedance to adjust the minimum frequency of the amplifier and the value of the second impedance to adjust the amplifier gain of the amplifier. The detailed construction and operation of the amplifiers 18 will be disclosed subsequently in connection with FIG. 8.

The outputs from each of the amplifiers 18 are introduced to a sample-and-hold circuit 32 which operates to sample all of the 32 amplifiers 18 simultaneously as to the outputs of the amplifiers and to process the simultaneously obtained outputs in sequence. This occurs on a cyclic basis. The simultaneous sampling of the outputs of the amplifier offers certain advantages. This allows the outputs of different amplifiers to be compared on a real time basis to provide information which cannot be provided by each amplifier alone. For example, a number of the 32 amplifiers may be providing indications of frequency and voltage amplitudes at different strategic terminals connected to particular amplifiers. It is desirable for the outputs from these amplifiers to be determined and measured simultaneously in order to provide a proper over-all indication of the sleep apnea of the patient.

The signals from the sample-and-hold circuit 32 are introduced to a multiplexer 34 which provides for the sequential transfer of the outputs from the successive amplifiers 18 to an analogto-digital converter 36. The digital signals from the converter 36 are then introduced to a data buffer 38 and from the buffer to the microprocessor 28. The microprocessor 28 then transfers the transferred data to a communication port from which data can be transferred to the host. The signals from the communication port 24 can then be transferred to a plurality of different kinds of communication interfaces, for example, the signals can be transferred to a digital subscriber line (DSL) or to a modem in a wireless unit or to a Bluetooth unit.

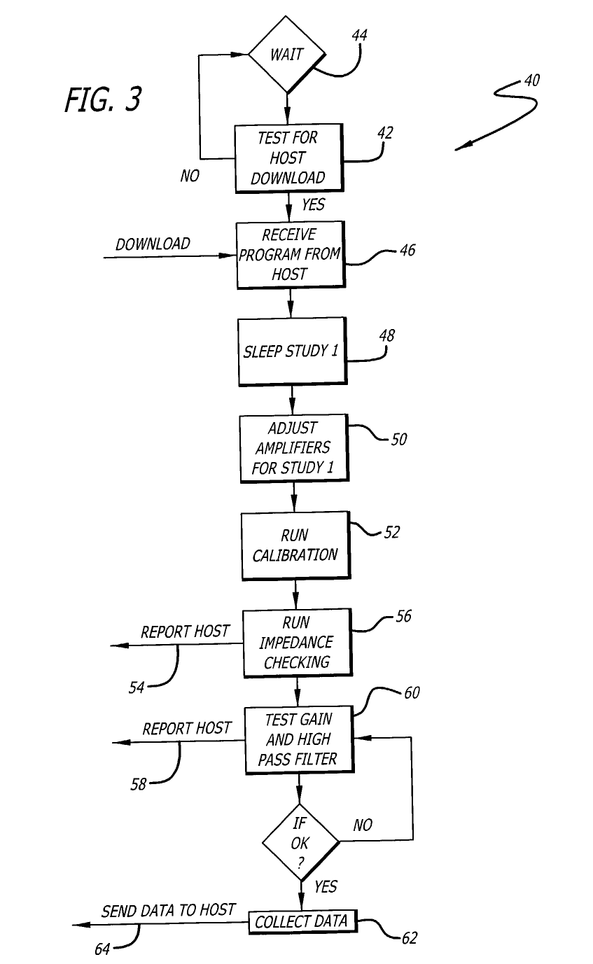

FIG. 3 is a flow chart generally indicated at 40 and showing a plurality of successive steps in the operation of each of the programmable solid state recorder (PSSR) 10 regardless of the individual ones of the physiological signals that are being processed by each of the amplifiers. At a first step 42, a test is made to determine whether the instructions for the operation of the amplifiers have been downloaded by the host through the microprocessor 28 to the amplifiers. This test may be performed on a sequential basis. If the answer is no, the processing is returned to a wait position 44. If the answer is yes, the PSSR 10 receives a download of a program from the host 24 (see 46). Assume that the PSSR 10 is to be downloaded to provide a sleep study in connection with a determination of sleep apnea. This is indicated at 48 in FIG. 3. The programmable solid state recorder (PSSR) 10 is then adjusted (see 50) to provide the sleep study. This adjustment may be in the adjustment of the first and second impedances (described previously and to be specified subsequently in connection with the embodiment of the amplifier shown in FIGS. 8-1 and 8-2) and in allocation of amplifiers to particulars transducers.

A calibration is then made of the amplifier (see 52) and any characteristics in the amplifier are then adjusted to provide for a passing of the calibration test. This calibration may be provided to the amplifiers on a sequential basis. The results of the calibration are then reported to the host as indicated at 54 in FIG. 3. A check is then made of the impedances (56) at the different terminals in the amplifier. The results of the impedance checks are reported to the host as at 58. A check 60 is then made of a gain and high pass filter (to be discussed in connection with FIG. 8). If the minimum frequency of the high pass filter is not at the desired value, the value of the adjustable impedance is adjusted to provide the proper value. If the gain is not within the particular upper and lower limits, the gain is adjusted (to be measured in connection with FIGS. 12 and 14) as discussed above. These gain and high pass filter tests and adjustments are indicated at 60 in FIG. 3. When the proper adjustments in the impedances have been made, the data from the amplifier is transmitted to a programmable solid state recorder (PSSR) 10 as indicated at 62 in FIG. 3. The programmable solid state recorders (PSSR) 10 are shown in FIGS. 5, 6 and 7. If requested by the host, this data may also be transmitted to the host as indicated at 64 in FIG. 3.

FIG. 4 is a circuit diagram showing in block form the construction of one of the amplifiers 18. The amplifier 18 receives inputs from three (3) terminals 70, 72 and 74. The terminal 70 constitutes a recording terminal. It receives the signals from one of the terminals such as the terminals 12 and 14 shown in FIG. 1. The terminal 72 provides a reference voltage terminal and the terminal 74 provides a patient's ground. The terminals 70, 72 and 74 are connected to a high pass filter and amplifier protection circuit 76. The high pass filter in the circuit 76 passes signals through a range of frequencies as high as approximately one thousand hertz (1 KHz). The circuit 76 is differential. This means that the circuit 76 will pass operational signals of interest but will reject noise. The circuit 76 includes a protection stage which limits the amplitude of the signals passing through the circuit 76. Applicant believes that he may be the first to provide a circuit, with the features provided by the circuit 76, in a system for acquiring the physiological signals of a patient.

The output from the circuit 76 is introduced to a gain stage 78. This gain stage also constitutes a differential amplifier so that it provides an additional rejection of noise. The gain stage provides a particular gain such as a gain of 10. The signals from the gain stage 78 are then introduced to a high pass filter 80. The high pass filter includes a capacitor having a fixed value and an impedance (e.g. a resistor) having an adjustable value. The impedance may be provided with 4 different values controlled by a pair of binary signals providing for a selection of one of the four (4) values. The relationship between the minimum frequency of the signals passing through the filter 80 at the different binary values is shown in a chart 81 below the filter. As will be seen, 4 binary values (represented by 2 binary signals) are shown in the first 2 columns where the values of the binary bits are indicated. The third column represents the minimal frequency of the signals passing through the filter 80. As will be seen, the minimal frequency may be 0, 0.01, 0.1 and 1 hertz depending upon the particular binary value selected.

The signals from the high pass filter 80 pass to a gain stage 82. The gain stage 82 includes a chopper stage which provides the gain stage with a stable DC reference. This tends to stabilize the DC gain provided by the stage. The stage 82 also includes a circuit which operates to adjust the value of an impedance in the stage so as to maintain the gain of the stage between particular maximum and minimum limits. A digital control similar to that shown for the high pass filter 80 and described above may be provided for the gain stage 82. This digital control is shown in a chart 83 below the gain stage 82. The digital control is provided by two (2) binary bits. As will be seen from the chart, gains of 50, 100, 500 and 1000 are respectively provided by adjusting the value of an impedance (e.g. a resistor) in the gain stage 82 when binary values of 00, 01, 10 and 11 are respectively provided for the binary control. More control lines would provide better resolution for gain adjustment.

The fifth stage in the amplifier 18 is a low pass filter 84 which reduces the frequency range from approximately 1000 hertz to approximately 100 hertz. The frequency reduction is obtained by providing three (3) successive filters each providing a decibel correction of approximately 40 db for a total correction of 120 db. However, the total db correction at 100 hertz is only approximately three (3) decibels. The low pass filter 84 is designed to preserve the original phase relationship of the signals at and below 100 hertz. This is important in providing reliable information concerning the physiological signals being measured. This is particularly important when phases of different signals are being compared and for time domain measurements. The signals from the low pass filter 84 are introduced to a driving amplifier 86 which may be of a conventional construction.

FIG. 5 illustrates a system, generally indicated at 90, in which the system (10) (PSSR) shown in FIG. 2 and including the amplifier 18 can operate. The system 90 includes a central archive and study evaluation center 92. The central archive and study evaluation center 92 may be considered as a host and is connected to the programmable solid state recorder (PSSR) 10 in FIG. 2. The central archive and study evaluation center 92 may be connected by digital subscriber lines (DSL) 94 to a pair or a number of programmable solid state recorders (PSSR) 96 and 98. Each of the recorders 96 and 98 may be considered to correspond to one of the amplifiers 18.

Each of the recorders 96 and 98 can send data (1) periodically to the central archive and study evaluation center 92 or (2) to the central archive and study evaluation center when its task has been completed or (3) to the central archive and study evaluation center when the recorder is queried by the archive central and study evaluation center. The central archive and study evaluation center 92 assesses the data from each of the recorders 96 and 98 to determine if the recorders are operating properly. If the central archive and study evaluation center 92 determines that one of the recorders 96 and 98 is not operating properly, the archive sends a signal to the recorder that the recorder is not operating properly. The recorder then makes an adjustment in its operation to satisfy the requirements of the central archive and study evaluation center. This is shown schematically in the charts 54, 58, and 64 in FIG. 3 and has been described in detail previously.

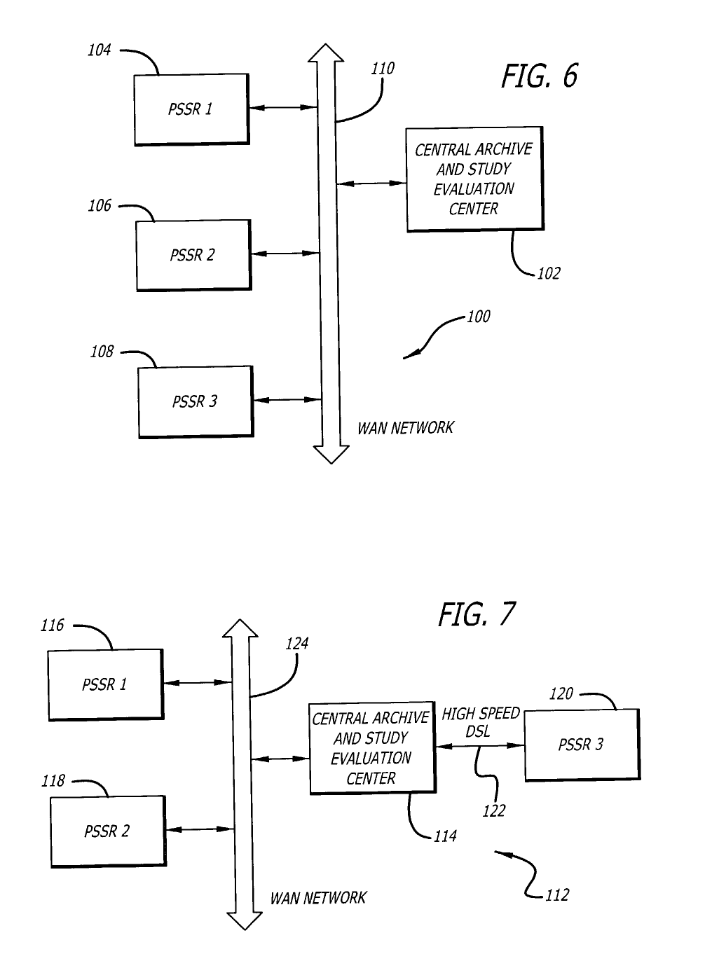

FIG. 6 illustrates another system, generally indicated at 100, similar to the system 90 in FIG. 5. The system 100 includes a central archive and study evaluation center 102 and recorders 104, 106 and 108 each corresponding to one set of 32 amplifiers 18 in FIG. 2. The central archive and study evaluation center 102 and the recorders 104, 106 and 108 are connected by a high speed communication port 110 which may be wireless. The system 100 in FIG. 6 has all of the advantages of the system 90 shown in FIG. 5 and described above.

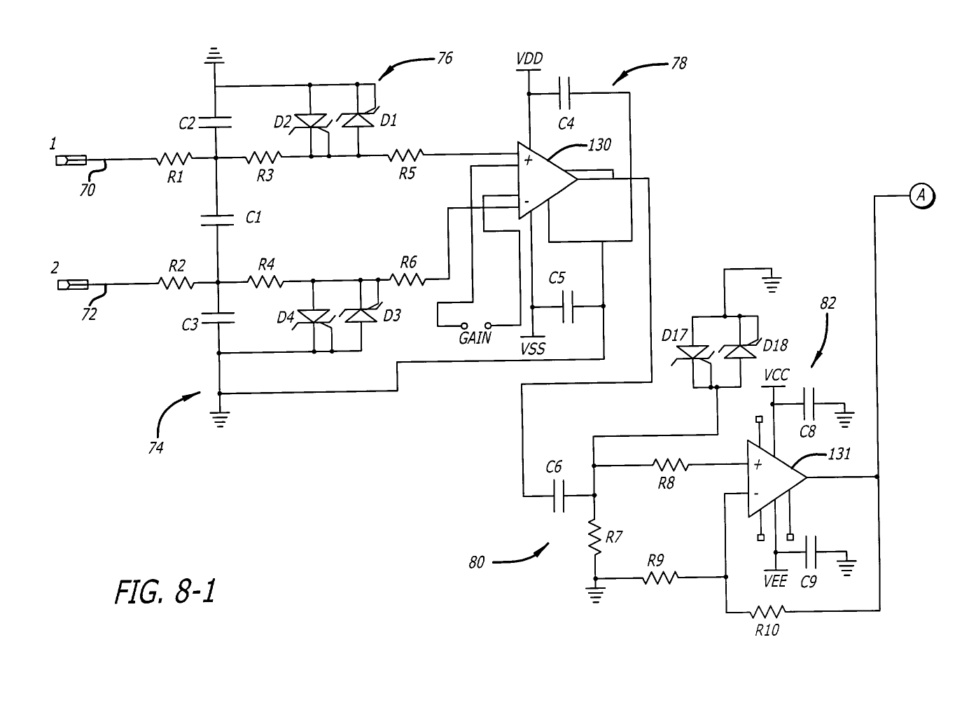

FIG. 7 includes a system, generally indicated at 112, which constitutes a combination of the systems shown in FIGS. 5 and 6. The system 112 includes a central archive and study evaluation center 114 and recorders 116, 118 and 120. The central archive and study evaluation center 114 may be connected to the recorder 120 by the digital subscriber line (DSL) 122 and may be connected to the recorders 116 and 118 by a high speed communication port 124 to provide a wireless communication between the archive and the recorder.

FIGS. 8-1 and 8-2 provide is a circuit diagram showing in detail the construction of one of the amplifiers 18. As previously indicated, all of the amplifiers 18 may have the same construction except that the value of a resistor R₇ may have a different one of 4 adjustable values than the value of that resistor in other ones of the amplifiers. This difference in values is indicated by the chart 81 in FIG. 4. A second exception is that the value of a resistor R₁₀ may have a different one of 4 adjustable values than the values of that resistor in other ones of the amplifiers. This difference is indicated by the chart 83 in FIG. 4.

The recording terminal 70 and the reference terminal 72 in FIG. 4 are respectively introduced to resistors R₁ and R₂ in the input high pass filter and amplitude protection 76, the stage also being shown in FIG. 8-1. The resistors R₁ and R₂ are respectively in series in FIG. 8-1 with capacitors C2 and C3, which are connected to the ground 74 (also shown in FIG. 4). The resistors R₁ and R₂ are also respectively in series with resistors R₃ and R₅ and with resistors R₄ and R₆. Parallel zener diodes D1 and D2 are connected between ground and the terminal common to the resistors R₃ and R₅. In like manner, zener diodes D3 and D4 are connected between ground and the terminal common to the resistors R4 and R6.

As will be seen, the stage 76 in FIG. 4 is a high pass filter. Because of this, noise is substantially eliminated. Furthermore, the stage passes signals through a frequency range to a frequency of approximately 1000 hertz. Signals above this frequency are passed by the capacitors C2 and C3 in FIG. 8-1 to ground. Furthermore, the amplitudes of the signals passing through the amplifier are limited by the zener diodes D1 and D2 and the zener diodes D3 and D4, all of which break down above a limiting voltage and provide a low impedance to ground. Limiting the voltage from the high pass filter 76 is advantageous because it facilitates the operation of the amplifier in processing the signals quickly.

The values of the components in the high pass filter 76 in FIGS. 4 and 8 may be as follows:

| Component | Value |
|---|---|
| R₁ | 1K |
| R₂ | 1K |
| R₃ | 10K |
| R₄ | 10K |
| R₅ | 10K |
| R₆ | 10K |
| C1 | 47nF |
| C2 | 68pF |
| C3 | 68pF |

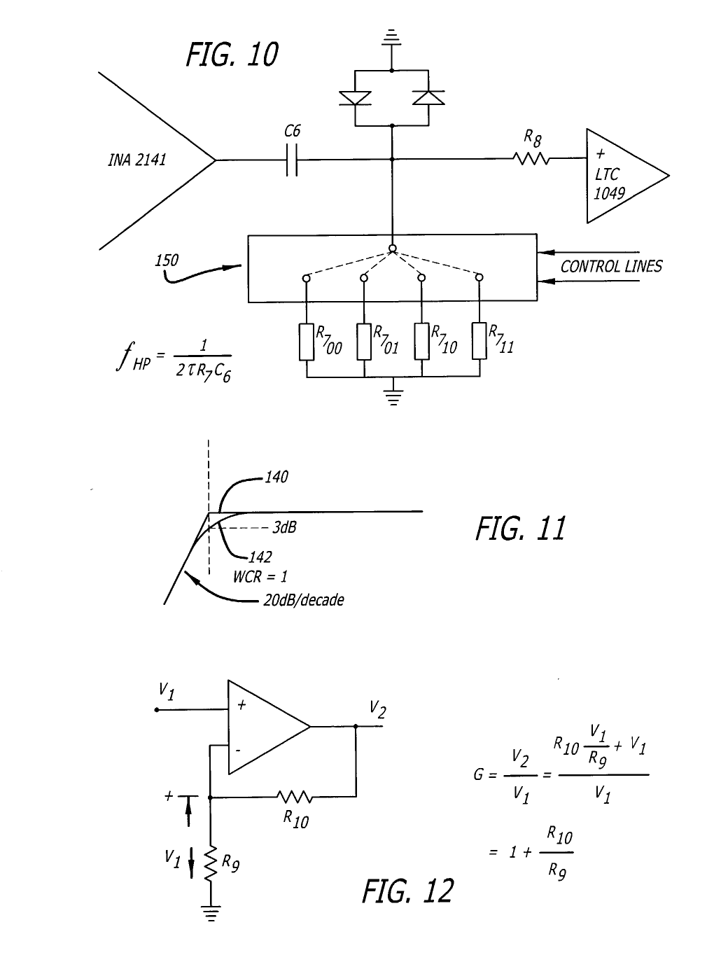

The outputs of the stage 76 are introduced to input terminals of an amplifier 130 included in the very high mode rejection differential amplifier stage 78 in FIG. 4. The amplifier 130 receives a positive voltage VDD and a negative voltage VSS in FIG. 8-1. Capacitors C4 and C5 respectively having values of 0.01 μF are included in the stage 78. The output of the amplifier 130 is introduced to the high pass filter 80 in FIG. 4. The filter 80 in FIG. 8-1 (1.77 M) includes a capacitor C6 (0.18 μF) and a resistor R₇ (1.77 M) connected in series to ground. The capacitor C6 passes signals at high frequencies to the resistor R₇ in FIG. 8-1 and blocks the passage of signals at low frequencies. The resistor R₇ can have four (4) different values as will be described subsequently in connection with FIG. 10. These four (4) different values provide for the four (4) different responses shown in the chart 81 in FIG. 4.

The output signals across the resistor R₇ in FIG. 8-1 are introduced to a resistor R₈ having a value of 499 ohms. The resistors R₉ and R₁₀ are connected to input terminals of a chopper 131 in FIG. 8. The chopper 131 is included in the gain stage 82 in FIG. 4. The chopper 131 operates to maintain a stable DC reference. The chopper is connected between a positive voltage VCC and a negative voltage VEE. Capacitors C8 and C9 are respectively connected between the voltage VCC and ground and between the voltage VEE and ground. Each of the capacitors C5 and C6 may have a value of approximately 0.01 μF.

Zener diodes D17 and D18 are respectively connected to ground from the terminal common to the capacitor C6 and the resistor R₈. The zener diodes D17 and D18 limit the voltage in the chopper 131. By maintaining the voltage across the resistor R₇ within particular limits, as a result of the inclusion of the zener diodes D17 and D18, the capacitor C6 is able to discharge to ground (which constitutes a stable reference) within a relatively short period of time. This is desirable in maintaining the same characteristics for the signals at the output of the capacitor C6 as the characteristics of the signals at the input to the capacitor.

The output of the chopper 131 is introduced to the low pass filter 84 in FIG. 4. The low pass filter 84 is provided with three (3) stages each having an identical construction and each providing an attenuation of approximately 40 decibels for a total attenuation of 120 db. In this way, the signals having a frequency above 100 hertz are eliminated and the signals at 100 hertz are provided with an attenuation of only 3 db. One of the three (3) stages in FIG. 8-2 may include a pair of resistors R₂₁, and R₂₂ (each having a value of approximately 100 kilohms), between the output of the chopper 131 and the input of an amplifier 132. A capacitor C19 having a value of approximately 12,000 pf extends electrically between the input terminal of the amplifier 132 and ground is connected to the output of the amplifiers. A capacitor C21 having a value of approximately 12,000 pf is connected between the output of the amplifier 132 and the terminal common to the resistors R₂₁, and R₂₂. One terminal of the amplifier 132 receives the positive voltage VCC and another terminal of the amplifier receives the negative voltage VEE. A VEE capacitor C20 having a value of approximately 0.1 μF is connected between the VCC and ground and the VEE voltage terminal and ground.

In addition to providing an attenuation of approximately 40 db, the low pass filter discussed above has another significant advantage. It maintains the phase relationship between the signals at the different frequencies even as it is eliminating the signals above approximately 100 hertz in frequency. As will be appreciated, it is important to maintain the phase relationship between the different frequencies to approximately 100 hertz in order to be able to determine differential measurements between different signals from the patient's body.

FIG. 10 shows the capacitor C6 and the resistor R₇ (FIG. 8), both of which define the high pass filter 80 in FIG. 4. The filter 80 is well known in the prior art but not for the purposes described in this application. The operation of this filter may be defined by the following equation:

$${f}_{HP} = \frac{1}{{2}_{π} {R}_{7} {C}_{6}} where$$

f_{HP}=the frequencies of the signals passed by the high pass filter 80;

R₇=the value of the resistor R₇; and

C₆=the value of the capacitor C6. With the value of the capacitor C6 constant, the following relationship exists:

f_{HP01}=0.01 hertz and R_{7 01} has a value of R;

f_{HP10}=0.1 hertz and R_{7 10} has a value of 10R; and

f_{HP11}=1 hertz and R_{7 11} has a value of 100R. In the above equations the frequencies f_{HP01}, f_{HP10} and f_{HP11} correspond to the second, third and fourth rows in the chart 81 in FIG. 4. FIG. 10 also shows a multiplexes switch 150 which can be operated in accordance with the operation of a multiplexer (not shown) so that a movable contact in the switch will provide a connection to any selected one of the resistors R_{7 00}, R_{7 01}, R_{7 10} and R_{7 11}. The multiplexer switch 150 has a stationary contact connected to the capacitor C6.

FIG. 11 indicates an attenuation of signals provided by the filter 80. As will be seen, an attenuation is provided below a particular frequency such as 0.01 hertz, 0.1 hertz and 1.0 hertz (depending upon the value of the resistor R₇) as in the equations indicated in the previous paragraph. As indicated in FIG. 11, a curve 140 indicates a desired attenuation at one of the cut off frequencies such as 0.01 hertz, 0.1 hertz and 1.0 hertz. Curve 142 indicates a curve which is actually obtained. In this curve, an attenuation of 3 db is provided at the cut-off frequency such as 0.01, 0.1 and 1.0 hertz and the attenuation increases at frequencies below the cut-off frequency.

FIG. 12 is a simplified circuit diagram showing the chopper 131 and the resistors R₉ and R₁₀ in FIG. 8. In FIG. 12, the resistor R₉ is a constant and the resistor R₁₀ is adjustable.

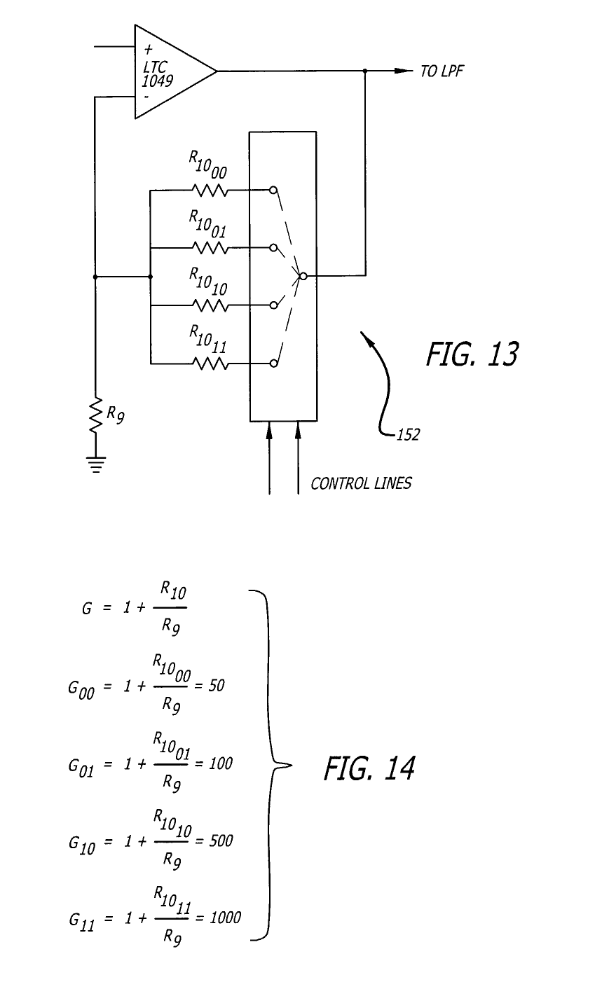

FIG. 13 shows the equation for determining the gain in the chopper 130. This is indicated by the equation:

$$G = 1 + \frac{{R}_{10}}{{R}_{9}} , where$$

$$G = the gain$$

For the binary values indicated in the chart 83 in FIG. 4, the gain may be indicated as follows:

$$G00 = 1 + \frac{{R}_{10 \_ 00}}{{R}_{9}} = 50$$

$$G01 = 1 + \frac{{R}_{10 \_ 01}}{{R}_{9}} = 100$$

$$G10 = 1 + \frac{{R}_{10 \_ 00}}{{R}_{9}} = 500$$

$$G11 = 1 + \frac{{R}_{10 \_ 01}}{{R}_{9}} = 1000$$

The circuitry shown in FIG. 12 operates on a closed loop basis to adjust the value of R₉ constantly so that an optimal value of gain is always provided. A gain of about 100 would be optimal. However, the value of the gain is maintained in the region of about 80% of the A/D converter full scale so that the value of the gain will not exceed the full scale of the analog-to-digital converter. This provides flexibility in the determination and maintenance of the gain.

FIG. 12 also shows a switch 152 having a stationary contact and four contacts indicated by broken lines as being engaged by a movable contact. The four (4) contacts are respectively connected to the resistors, R_{10 00}, R_{10 01}, R_{10 10} and R_{10 11}. The position of the movable contact is determined by a multiplexer. If more control lines are provided, each of the frequency and gain selections will have more than four steps and resolution will be enhanced, especially for the gain stage.

FIG. 14 shows the gain which is provided when the movable contact in the switch 152 contacts each individual one of the resistors, R_{10 00}, R_{10 01}, R_{10 10} and R_{10 11}.

Although this invention has been disclosed and illustrated with reference to particular preferred embodiments, the principles involved are susceptible for use in numerous other embodiments which will be apparent to persons of ordinary skill in the art. The invention is, therefore, to be limited only as indicated by the scope of the appended claims.

What is claimed is:

1. In combination for determining individual ones of a plurality of physiological conditions of a patient,
an amplifier constructed to measure and process the physiological rights of the patient and to provide analog signals representative of the measurements,
an analog-to-digital converter operationally coupled to the amplifier for converting the analog signals to digital signals,
a microprocessor for selecting individual ones of the physiological signals to be measured and processed by the amplifier and for adjusting characteristics of the amplifier to measure and process the selected physiological signals, the microprocessor being operative to receive the digital signals from the converters, and
a member operatively coupled to the microprocessor for receiving the digital signals from the microprocessor.

2. In a combination as set forth in claim 1,
the physiological signals including signals having particular frequency ranges, and the microprocessor being operative to select an individual one of the particular frequency ranges to be measured and processed by the amplifier.

3. In a combination asset forth in claim 1,
the physiological signals including signals having particular amplitude gains and
the microprocessor being operative to select an individual one of the particular amplitude gains for the signals measured and processed by the amplifier.

4. In a combination as set forth in claim 2 wherein
an impedance included in the amplifier is provided with different values to provide the particular frequency ranges and wherein
the impedance is provided by the microprocessor with an individual one of the impedance values to select an individual one of the frequency ranges for the signals.

5. In a combination as set forth in claim 3 wherein
a first impedance is included in the amplifier and is provided with different values to provide the signals with a particular high pass frequency and wherein
a second impedance is provided with an individual one of different values to provide the amplifier with the particular gain.

6. In a combination as set forth in claim 2,
the physiological signals including signals having particular amplitude gains, and
the microprocessor being operative to select an individual one of the particular amplitude gains for the signals measured and processed by the amplifier.

7. In a combination as set forth in claim 4,
the physiological signals including signals having particular amplitude gains;
the microprocessor being operative to select an individual one of the particular amplitude gains for the signals measured and processed by the amplifier, and
a first impedance included in the amplifier and provided with different values to provide the signals with a particular frequency,
the impedance being provided with an individual one of the different values to provide the amplifier with the particular frequency.

8. In combination for determining individual ones of a plurality of physiological conditions of a patient,
a plurality of amplifiers each constructed to measure and process the physiological conditions of the patient and to provide analog signals representative of the measurements,
a microprocessor for selecting individual ones of the physiological signals to be measured and processed by each of the amplifiers,
each of the amplifiers being operatively coupled to the microprocessor to measure and process the individual ones of the physiological conditions selected by the microprocessor to be measured and processed, and
a sample-and-hold circuit operatively coupled to the amplifiers to provide for a simultaneous measurement of the selected physiological signals in the amplifiers and to process the physiological signals sequentially.

9. In a combination as set forth in claim 8,
the signals produced by the sample-and-hold circuit being analog signals, and
an analog-to-digital converter responsive to the analog signals from the sample-and-hold circuit for converting the signals to digital signals.

10. In a combination as set forth in claim 9,
an output stage,
the microprocessor being connected to the converter for receiving the digital signals from the converter for each of the amplifiers and for introducing the digital signals to the output stage.

11. In a combination as set forth in claim 8 wherein
each of the amplifiers includes a stage with different values of an impedance and where the different values of the impedance affect the gain of the amplifier and wherein
the microprocessor determines the gain of the signals from each amplifier and provides for an adjustment in the value of the impedance to maintain the gain of the amplifier within the particular limits.

12. In a combination as set forth in claim 8 wherein
each of the amplifiers includes a stage with different values of an impedance and wherein the different values of the impedance for the amplifier affect the frequency of the signals provided by the amplifier and wherein
the microprocessor determines the frequency of the signals to be provided by each of the amplifiers and provides for an adjustment in the value of the impedance to provide the determined frequency.

13. In a combination as set forth in claim 9,
an output stage,
the microprocessor being connected to the converter for receiving the digital signals from the converter for each of the amplifiers and for introducing the digital signals to the output stage.

14. In a combination as set forth in claim 11 wherein
each of the amplifiers includes a stage with different values of an impedance and wherein the different values of the impedance in the amplifier affect the frequency of the signals provided by the amplifier and wherein
the microprocessor determines the frequency of the signals to be provided by each of the amplifiers and provides for an adjustment in the value of the impedance to produce the physiological signals within a particular frequency range.

15. In a combination as set forth in claim 13 wherein
each of the amplifiers includes a stage with different values of a first impedance and where the different values of the first impedance affect the frequency of the amplifier and where
the microprocessor provides for an adjustment in the value of the first impedance to provide the frequency of the signals within a particular frequency range and wherein
each of the amplifiers includes a stage with different values of a second impedance and wherein the different values of the second impedance affect the gain of the signals provided by the amplifier and wherein
the microprocessor determines the frequency of the signals to be provided by each of the amplifiers and provides for an adjustment in the value of the second impedance to provide the determine gain.

16. In combination for determining individual ones of a plurality of physiological conditions parameters of a patient,
a plurality of amplifiers each operative to provide measurements and processing of the physiological conditions of a patient,
input circuitry operative to select an operation of each of the amplifiers to measure and process individual ones of the physiological conditions,
sample-and-hold circuitry operative to sample the amplifiers simultaneously to obtain physiological signals representative of the individual ones of the physiological conditions selected for the amplifiers and to process the physiological signals from the different amplifiers sequentially, and
output circuitry for analyzing the characteristics of the processed signals.

17. In a combination as set forth in claim 16,
a microprocessor for providing for the sampling of the amplifiers on a simultaneous basis and the measuring and processing of the sampled signals on a sequential basis.

18. In a combination as set forth in claim 17,
the physiological conditions being measured in the characteristics of the signals produced by the amplifiers,
the microprocessor being operative to adjust the gain of each of the amplifiers to be between particular lower and upper limits in accordance with the selection provided by the input circuitry and to adjust the frequency of the signals in accordance with the characteristics of the physiological conditions being measured, and
the microprocessor being operative to provide the signals from the amplifiers after the adjustment in the gain and the frequency of the signals.

19. In a combination as set forth in claim 18 wherein
the physiological conditions are provided in the characteristics of the signals produced by the amplifiers and wherein
the microprocessor is operative to adjust the frequency range of each of the amplifiers in accordance with the selection of the physiological conditions by the input circuitry and wherein
the amplifier is operative to provide the signals to the sample-and-hold circuit after the adjustment of the frequency range of the signals in the amplifier.

20. In a combination as set forth in claim 18 wherein
the adjustment in the gain in each of the amplifiers to be between the particular upper and lower limits is provided by adjusting the value of an impedance in the amplifier.

21. In a combination as set forth in claim 19 wherein
the adjustment in that frequency range of the signals in each of the amplifiers is provided by adjusting the value of an impedance in the amplifier.

22. In a combination as set forth in claim 18 wherein
the physiological conditions are measured in the characteristics of the signals produced by the amplifiers and wherein
the microprocessor is operative to adjust the frequency range of each of the amplifiers in accordance with the selection of the physiological conditions by the input circuitry and wherein
the amplifier is operative to provide the signals to the sample-and-hold circuit after the adjustment of the frequency range of the signals in the amplifier.

23. In combination for determining individual ones of plurality of physiological conditions of a patient in each of a plurality of channels,
a host for indicating individual ones of the physiological conditions of the patient in each of the channels,
a plurality of amplifiers each disposed in an individual one of the channels,
a microprocessor responsive to the indications from the host of the individual ones of the physiological signals for the channels to provide the physiological signals for the channel,
the amplifiers being operative upon the provision of the individual ones of the physiological signals for the channel to provide output signals indicative of the physiological condition,
the microprocessor being responsive to the signals from the amplifiers for introducing the signals to the host.

24. In a combination as set forth in claim 23,
a plurality of terminals each connected to the patient at a particular position on the patient and each operative to provide signals representative of the physiological status condition of the patient upon the provision of the individual ones of the physiological signals for the channel.

25. In a combination as set forth in claim 23,
the physiological signals for each of the amplifiers being related to the frequency range of the signals in the amplifier.

26. In a combination as set forth in claim 23,
the microprocessor being responsive to the gain of the output signals for adjusting the gain to be within particular upper and lower limits.

27. In a combination as set forth in claim 24 wherein
one of the physiological signals for each of the amplifiers is related to the frequency range of the signals in the amplifiers and wherein
the microprocessor is responsive to the gain of the signals from each of the amplifiers for adjusting the gain to be within particular limits.

28. In a combination for determining individual ones of a plurality of physiological conditions of a patient in each of a plurality of channels,
a plurality of amplifiers each constructed to provide signals indicative of individual ones of the different physiological conditions,
a plurality of terminals each connected to an individual one of the amplifiers to provide to the amplifier signals indicative of the individual ones of the physiological conditions for amplification of the signals by the amplifier, the terminals being adapted to be applied to the body of the patient,
a host for indicating the physiological conditions to be determined by each of the amplifiers, and
a microprocessor responsive to signals from the host for introducing the signals to the amplifiers to control the operation of each of the amplifiers in providing signals in an individual range of frequencies dependent upon the physiological conditions being measured by the amplifier where the characteristics of the signals from the amplifier are representative of the physiological conditions being provided by the amplifier.

29. In a combination as set forth in claim 28 wherein
the microprocessor activates the amplifiers simultaneously to obtain signals simultaneously on a real time basis from the amplifiers and wherein
circuitry is provided for processing the signals sequentially from the amplifiers.

30. In a combination as set forth in claim 29 wherein
the microprocessor provides for the amplifiers to maintain the gains of the amplifiers within particular minimum and maximum limits.

31. In a combination as set forth in claim 29 wherein
sample-and-hold circuitry is provided to receive the signals produced simultaneously on a real time basis by the amplifiers and to provide for the passage of the received signals on a sequential basis from the amplifiers and wherein
the signals passing sequentially from the sample-and-hold circuitry are processed and the processed signals are introduced to the host.

32. In a combination as set forth in claim 29 wherein
sample-and-hold circuitry is provided to receive the signals produced simultaneously on a real time basis by the amplifiers and to provide for the passage of the received signals on a sequential basis from the amplifiers and wherein
the signals passing sequentially from the sample-and-hold circuitry are processed and the processed signals are introduced to the host.

33. In combination as set forth in claim 29 wherein
the microprocessor provides for the amplifiers to maintain the gains of the amplifiers within particular minimum and maximum limits and wherein.
sample-and-hold circuitry is provided to receive the signals produced simultaneously on a real time basis by the amplifiers and to provide for the passage of the received signals on a sequential basis from the amplifiers and wherein
the signals passing sequentially from the sample-and-hold circuitry are processed and the processed signals are introduced to the host.

34. In combination for determining individual ones of a plurality of physiological conditions for a patient in each of a plurality of channels,
a plurality of amplifiers each constructed to provide signals indicative of individual ones of the plurality of different physiological conditions, each of the amplifiers including a high pass filter and a gain control circuit,
a plurality of terminals each connected to an individual one of the amplifiers to provide to the amplifiers signals indicative of the individual ones of the physiological conditions from the amplifiers, the terminals being adapted to be applied to the body of the patient,
a microprocessor associated with the amplifiers for adjusting the operation of the high pass filters in each of the amplifiers in accordance with the physiological conditions to be indicated by the amplifiers and for adjusting the gain of the amplifiers to be within particular minimum and maximum limits, and
a host for providing instructions to the microprocessor to control the adjustments provided by the microprocessor in the high pass filter in each of the amplifiers.

35. In a combination as set forth in claim 33 wherein
the microprocessor adjusts the gain of each of the amplifiers to be within particular minimum and maximum limits and whereon
the host provides for the microprocessor to obtain from the amplifiers, after the high process filter and the gain control have been adjusted in accordance with the instructions from the host, signals having characteristics of the physiological conditions to be determined by the amplifiers.

36. In a combination as set forth in claim 33,
the high pass filter in each of the amplifiers being constructed to pass signals through a first range of frequencies,
each of the amplifiers also including stages for reducing the frequency of the signals from the first range to a range of frequencies in which the physiological conditions occur.

37. In a combination as set forth in claim 33 wherein
the amplifiers are constructed from a plurality of components and wherein
the amplifiers have the same construction with the same component values regardless of the parameters being determined by the amplifiers with the exception of variations in a value of an impedance in the high pass filter and variations in a value of an impedance in the gain control circuit.

38. In a combination as set forth in claim 34,
the high pass filter in each of the amplifiers being constructed to pass signals through a first range of frequencies,
each of the amplifiers also including stages for reducing the frequency of the signals from the first range to a range of frequencies in which the physiological conditions occur and wherein
the amplifiers are constructed from a plurality of components and wherein the amplifiers have the same construction with the same component values regardless of the physiological conditions being determined by the amplifiers with the exception of variations in a value of an impedance in the high pass filter and variations in a value of an impedance in the gain control circuit.

39. In combination for determining individual ones of a plurality of physiological conditions of a patient,
a plurality of programmable recorders for indicating individual ones of the physiological conditions of the patient,
a central archive and study evaluation center for instructing each of the recorders to indicate individual ones of the physiological conditions of the patient,
a digital subscriber line,
first ones of the recorders being operative to communicate with the station through the digital subscriber line,
a high speed modem,
second ones of the recorders being operative to communicate with the station on a wireless basis through the modem,
each of the recorders being operative to transmit to the stations signals indicative of the physiological conditions being determined by the recorder in accordance with the instructions from the station,
the station being responsive to the signals from each of the recorders to determine if the recorder is operating properly and being operative, upon an improper operation of the recorders, to change the operation of the recorders to have the recorders operate properly.

40. In a combination as set forth in claim 38,
each of the recorders including an amplifier having adjustable characteristics,
the station being responsive to an improper operation of each of the recorders for adjusting the characteristics of the recorder to have the recorder operate properly.

41. In a combination as set forth in claim 38,
one of the adjustable characteristics in each of the recorders being the frequency range of the amplifier in each of the recorders,
the station being responsive to an improper operation of each of the recorders for adjusting the frequency characteristics of the amplifier in the recorder to have the recorder operate properly.

42. In a combination as set forth in claim 38,
one of the adjustable characteristics in each of the recorders being the gain of the amplifier in the recorder,
the station being responsive to an improper operation of each of the recorders for adjusting the gain of the recorder to have the recorder operate properly.

43. In a combination as set forth in claim 38,
a plurality of terminals each associated with an individual one of the recorders and each constructed to be connected to the patient's body to have the recorder provide a determination of individual ones of the physiological conditions of the patient.

44. In a combination as set forth in claim 39,
one of the adjustable characteristics being the frequency range of the amplifier in each of the recorders,
the station being responsive to an improper operation of each of the recorders for adjusting the frequency characteristics of the amplifier in the recorder to have the recorder operate properly.
another one of the adjustable characteristics in each of the recorders being the gain of the amplifier in the recorder,
the station being responsive to an improper operation of each of the recorders for adjusting the gain of the recorder to have the recorder operate properly.
a plurality of terminals each associated with an individual one of the recorders and each constructed to be connected to the patient's body to have the recorder provide a determination of individual ones of the physiological signals of the patient.

45. In a combination for determining individual ones of a plurality of physiological conditions of a patient,
a plurality of programmable recorders for indicating individual ones of the physiological conditions of the patient,
a central archive and study evaluation station for instructing each of the recorders to indicate individual ones of the physiological conditions of the patient,
a digital subscriber line,
first one of the recorders being operative to communicate with the station through the digital signal line,
a high speed modem,
second ones of the recorders being operative to communicate with the station on a wireless basis through the modem,
each of the recorders being operative to transmit to the station signals indicative of the physiological conditions being determined by the recorder in accordance with the instructions from the station.

46. In a combination as set forth in claim 44,
each of the recorders including an amplifier,
each of the amplifiers being programmable to adjust the frequency range of the signals provided by the amplifier and being programmable to adjust the gain of the amplifier.

47. In a combination as set forth in claim 44,
a plurality of terminals each associated with an individual one of the recorders and each constructed to be connected to the patient's body to have the recorder provide a determination of individual ones of the physiological conditions of the patient.

48. In combination as set forth in claim 45,
each of the amplifiers having an identical construction regardless of the physiological conditions to be determined by the associated recorder except for a first impedance having adjustable characteristics dependent upon the frequency range of the recorder and except for a second impedance having adjustable characteristics to provide a gain in the amplifier between upper and lower limits.

49. In a combination as set forth in claim 45,
a plurality of terminals each associated with an individual one of the recorders and each constructed to be connected to the patient's body to have the recorder provide a determination of individual ones of the physiological signals of the patient, and
each of the amplifiers having an identical construction regardless of the physiological conditions to be determined by the associated recorder except for a first impedance having adjustable characteristics dependent upon the frequency range of the recorder and except for a second impedance having adjustable characteristics to provide a gain in the amplifier between upper and lower limits.

50. In combination in an amplifier for determining individual ones of a plurality of physiological conditions of a patient,
means for providing input signals representing the individual ones of the physiological conditions,
a differential filter-amplifier for amplifying the input signals in a first range of frequencies and for rejecting noise,
a filter for providing signals in a range of frequencies reduced relative to the range of frequencies of the signals from the differential filter-amplifier, and
a stage providing an adjustable gain in the signals from the differential filter-amplifier to provide the gain within particular upper and lower limits.

51. In a combination as set forth in claim 49 wherein
a plurality of amplifiers including the first amplifier are provided and wherein
the construction of the amplifiers remains substantially constant regardless of the physiological conditions being determined by the amplifiers except that a first impedance is adjustable, in the stage providing the adjustable frequency and except that a second impedance is adjustable in the stage providing the adjustable gain, to adjust the frequency and gain of the signals passing through the stage in accordance with the physiological signals to be determined by the amplifiers.

52. In a combination as set forth in claim 47 wherein
the gain is adjustable on a binary basis to provide a number of different gains dependent upon the value of the binary bits.

53. In a combination as set forth in claim 49 wherein
the filter is adjustable on a binary basis to provide a number of different ranges of frequencies dependent upon the value of the binary bits.

54. In a combination as set forth in claim 49 wherein
the gain is adjustable on a binary basis to provide a number of different gains dependent upon the value of the binary bits and wherein
the filter is adjustable on a binary basis to provide a number of different ranges of frequencies dependent upon the value of the binary bits.

55. In combination for determining individual ones of a plurality of physiological conditions of a patient,
a filter having differential properties for receiving input signals representative of individual ones of the physiological conditions of the patient and for passing the signals in a first range of frequencies while reducing noise,
a differential amplifier for providing a further elimination of noise and a gain in amplification in the signals from the filter,
a gain stage for providing gain in the signals from the differential amplifier between particular maximum and minimum levels and for providing a stable D C reference, and
a low pass filter for passing the signals only in a reduced range of frequencies relative to the signals from the gain stage and for preserving the relative phases of the signals in the reduced frequency range.

56. In a combination as set forth in claim 54 wherein
the gain stage provides for a binary control in the gain provided in the gain stage to maintain the gain between the particular maximum and minimum limits.

57. In a combination as set forth in claim 54 wherein
an additional gain stage is provided and the additional gain stage includes a capacitor and includes members for limiting the amplitudes of the signals passing through the stage to provide for a rapid discharge of the capacitor to maintain the characteristics of the signals in the additional gain stage.

58. In a combination as set forth in claim 54 wherein
terminals are provided for coupling to the patient's body to provide signals having frequency ranges dependent upon the positioning of the terminals on the patient's body and wherein
the signals on the terminals are introduced to the filter.

59. In a combination as set forth in claim 57 wherein
the frequency of the signals on the terminals have a frequency range within approximately 100 hertz and wherein
the frequencies of the signals within the range of approximately 100 hertz are dependent upon the positioning of the terminals on the patient's body and wherein
the low pass filter passes the signals in the frequency range to approximately 100 hertz.

60. In a combination as set forth in claim 58 wherein the filter receiving the signals from the terminals passes the signals in a frequency range to approximately 1000 hertz.

61. In a combination as set forth in claim 59 wherein
an additional gain stage is provided and the additional gain stage includes a capacitor and includes members for limiting the amplitudes of the signals passing through the stage to provide for a rapid discharge of the capacitor to maintain the characteristics of the signals in the additional gain stage and wherein
terminals are provided for coupling to the patient's body to provide signals having frequency ranges dependent upon the positioning of the terminals on the patient's body and wherein
the signals on the terminals are introduced to the filter.

62. In combination for determining individual ones of a plurality of physiological conditions of a patient,
a low pass filter having differential properties for receiving input signals representative of individual ones of the physiological conditions of the patient and for passing the signals in a first range of frequencies while reducing noise,
a low pass filter providing a limit on amplitude for passing quickly the signals from the low pass filter,
a gain stage responsive to the signals from the low pass filter for providing a gain in the signals within particular maximum and minimum limits, and
a low pass filter for passing the signals from the gain stage only in a reduced range of frequencies relative to the signals from the gain stage and for preserving the relative phases of the signals in the reduced frequency range.

63. In a combination as set forth in claim 61 wherein
the gain stage includes an impedance having a variable value for providing the gain in the signals within the particular maximum and minimum levels.

64. In a combination as set forth in claim 61 wherein
the gain stage includes a chopper having properties for maintaining a stable D C reference in the gain stage.

65. In a combination as set forth in claim 61 wherein
the high pass filter includes an impedance having a variable value to adjust the frequencies of the signals from the high pass filter in accordance with the physiological conditions to be determined.

66. In a combination as set forth in claim 61,
an additional high pass filter disposed between the differential amplifiers and the gain stage and having a variable value to provide an adjustment in the frequency range of the signals passing through the filters, this adjustment being provided in accordance with the variations in the value of the impedance.

67. In a combination as set forth in claim 62 wherein,
the gain stage includes a chopper having properties for maintaining a stable D C reference on the gain stage and wherein.
the gain stage includes an impedance having a variable value to provide an adjustment in the gain to maintain the gain between the particular maximum and minimum limits and wherein
an additional high pass filter is disposed between the differential amplifiers and the gain stage and has a variable value to provide an adjustment in the frequency range of the signals passing through the filter, this adjustment being provided in accordance with the variations in the value of the impedance.

68. In combination for determining individual ones of a plurality of physiological conditions of a patient,
a low pass filter having differential properties for receiving input signals representative of individual ones of the physiological conditions of the patient in a first range of frequencies while reducing noise,
stages for controlling the amplitude and gain of the signals from the low pass filter and for providing a stable DC reference for a processing of the signals, and
a low pass filter for passing the signals only in a reduced range of frequencies relative to the range of frequencies of the signals passed by the high pass filter and for preserving the relative phases of the signals in the reduced frequency.

69. In a combination as set forth in claim 67 wherein
the low pass filter passes signals in a range of frequencies to approximately 1000 hertz and wherein,
the second low pass filter passes signals in a range of frequencies to approximately only 100 hertz.

70. In a combination as set forth in claim 67 wherein
a terminal is connected to the low pass filter and is constructed to be connected to the patient to provide signals to the low pass filter in a range of frequencies dependent on the position where the terminal is connected on the patient's body and wherein
the gain stage provides for a binary control in the gain provided in the gain stage to maintain the gain between the particular maximum and minimum limits and wherein
a capacitor is included in the gain stage and wherein the maintenance of the gain within the particular maximum and minimum limits provides for a fast discharge of the capacitor and wherein
a terminal is provided for coupling to the patient's body to provide signals having a frequency range dependent upon the positioning of the terminal on the patient's body and wherein
the signals on the terminal are introduced to the filter and wherein
the frequency of the signals on the terminal has a frequency range within approximately one hundred hertz (100) and the frequencies of the signals within the range of approximately 100 hertz are dependent upon the positioning of the terminal on the patient's body and wherein
the low pass filter passes the signals only in the frequency range to approximately 100 hertz.

71. In a combination as set forth in claim 67 wherein
the stages have a first impedance adjustable in value to control the range of frequencies in the signals passed in the stages and wherein
the stages have a second impedance adjustable in value to maintain the gain in the stages between particular maximum and minimum limits.

72. In a combination as set forth in claim 68 wherein
a terminal is connected to the low pass filter and is constructed to be connected to the patient to provide signals to the low pass filter in a range of frequencies dependent on the position where the terminal is connected on the patient's body, and wherein
the gain stage provides for a binary control in the gain provided in the gain stage to maintain the gain between particular maximum and minimum limits and wherein a capacitor is included in the gain stage and wherein the maintenance of the gain within the particular maximum and minimum limits provides for a fast discharge of the capacitor and wherein
a terminal is provided for coupling to the patient's body to provide signals having frequency ranges dependent upon the positioning of the terminal on the patient's body and wherein the signals on the terminal are introduced to the filter and wherein
the frequency of the signals on the terminal has a frequency range within approximately one hundred hertz (100) and the frequencies of the signals within the range of approximately 100 hertz are dependent upon the positioning of the terminal on the patient's body and wherein
the low pass filter passes the signals only in the frequency range to approximately 100 hertz and wherein
another high pass filter provided a limit on amplitude for passing the signals from the high pass filter quickly and wherein
stages are provided for controlling the amplitude and gain of the signals from the high pass filter and for providing a stable DC reference for facilitating the processing of the signals and wherein
the stages have a first impedance adjustable in value to control the range of frequencies in the signals passed in the stages and wherein
the stages have a second impedance adjustable in value to maintain the gain in the stages between particular maximum and minimum limits.

73. A method of determining individual ones of a plurality of physiological conditions of a patient, including the steps of:
downloading a program for a PSSR solid state recorder (PSSR) from a host,
adjusting the characteristics of the amplifier in accordance with the program from the host,
providing a calibration of the amplifier and adjusting the amplifier to meet calibration standards,
adjusting impedances in the amplifier in accordance with the program downloaded to the amplifier,
adjusting the frequency range of the amplifier in accordance with the program downloaded to the amplifier from the host,
testing and adjusting the gain of the amplifier to provide the gain within particular upper and lower limits when the calibration test has been completed, and
sending data from the amplifier to the host when the previous steps have been successfully completed.

74. A method as set forth in claim 72 wherein
the frequency range is adjusted by adjusting the value of an impedance in the amplifier.

75. A method as set forth in claim 72 wherein
the gain of the amplifier is adjusted on a binary basis to be within particular upper and lower limits by adjusting the value of an impedance in the amplifier and wherein
the gain is adjustable on a binary basis to provide a number of different gains dependent upon the number of binary bits.

76. A method as set forth in claim 71 where
a plurality of amplifiers are provided and wherein
the data is obtained simultaneously by the amplifiers in the plurality and wherein
the data obtained by the amplifiers is sent sequentially by the amplifiers to the host.

77. A method of determining individual ones of a plurality of physiological conditions of a patient, including the steps of:
downloading a program for a PSSR from a host,
testing and adjusting the gain of the amplifier to provide the gain within particular upper and lower limits,
adjusting the frequency range of the amplifier in accordance with the program downloaded to the amplifier, and
determining the physiological signals of the patient in accordance with the program provided to the amplifier from the host.

78. A method as set forth in claim 76 wherein
the amplifier is one of a plurality of amplifiers and wherein
programs are downloaded to the amplifiers from the host and
wherein the amplifiers provide data simultaneously for the physiological conditions from the patient in accordance with the downloading from the host and wherein
the amplifiers provide their data sequentially.

79. A method as set forth in claim 77 wherein
the amplifiers are tested sequentially for proper operation of the amplifiers in accordance with instructions from the host.

80. A method as set forth in claim 77 wherein
the amplifiers are calibrated sequentially in accordance with instructions from the host.

81. A method as set forth in claim 78 wherein
the amplifiers are calibrated sequentially in accordance with instructions from the host.

82. A method of determining individual ones of a plurality of physiological conditions for a patient including the steps of
downloading programs for a plurality of amplifiers from a host,
attenuating the signals in the amplifiers to a first range of frequencies,
producing particular gains in the amplifiers, and
attenuating the signals in the first range of frequencies to signals in a second range of frequencies lower than the first range of frequencies while maintaining the phases of the signals in the second range of frequencies.

83. A method as set forth in claim 81 wherein
the attenuation of the signals to the first range of frequencies is provided on a differential basis.

84. A method as set forth in claim 81 wherein
the gain of each of the amplifiers is limited within particular minimum and maximum values in accordance with the instructions from the host.

85. A method as set forth in claim 81 wherein
the lower cut-off limit of the signal frequencies of the amplifiers is adjusted in accordance with the physiological conditions to be determined by the amplifiers.

86. A method as set forth in claim 82 wherein
the gain of each of the amplifiers is limited within particular minimum and maximum values in accordance with the instructions from the host and wherein
the lower cut-off limit of the signal frequencies of the amplifiers is adjusted in accordance with the physiological conditions to be determined by the amplifiers.

87. A method of determining individual ones of a plurality of physiological conditions of a patient, including the steps of:
downloading instructions to a plurality of amplifiers from a host relating to the individual ones of the physiological conditions of the patient to be determined by each of the amplifiers,
adjusting the characteristics of each of the amplifiers to provide measurements of the physiological conditions of the amplifiers in accordance with the instructions from the host, and
providing the measurements in the amplifiers of the physiological conditions to be measured by the amplifiers in accordance with the instructions from the host.

88. A method as set forth in claim 86, including the steps of:
the measurement of the physiological conditions of the patient in the amplifiers being provided on a simultaneous basis, and
providing a sequential basis from the amplifiers the indications of the measurements of the physiological conditions in the amplifier.

89. A method as set forth in claim 86 wherein
the step of adjusting the characteristics of the amplifiers is performed on a sequential basis for the different amplifiers in the plurality.

90. A method as set forth in claim 86 wherein
the amplifiers are calibrated on a sequential basis.

91. A method as set forth in claim 86 wherein
terminals are applied to the body of the patent at strategic positions on the body of the patient to measure the physiological signals of the patient.

92. A method of determining individual ones of a plurality of physiological parameters, including the steps of:
introducing instructions from a host to each of a plurality of amplifiers to have the amplifier determine individual ones of the physical parameters of the patient,
simultaneously determining the individual ones of the physiological parameters in each of the amplifiers in accordance with the instructions from the host, and
sequentially providing outputs indicating the individual ones of the physiological parameters of the amplifiers in the plurality.

93. A method as set forth in claim 90 wherein
the amplifiers in the plurality are calibrated on a sequential basis before the physiological conditions of the patient are determined by the amplifiers.

94. A method as set forth in claim 91 wherein
tests are sequentially performed on the amplifiers to provide for the proper operation of the amplifiers before the measurements are made of the physiological conditions of the patient.

95. A method as set forth in claim 91 wherein
tests are sequentially performed on the amplifiers to provide for the proper operation of the amplifiers before the measurements are made of the physiological conditions of the patient.
